(19) United States
(12) Patent Application Publication
(10) Pub. No.: US 2005/0197286 A1
(43) Pub. Date: Sep. 8, 2005

(54) Method for control of depression using c terminal growth hormone (gh) fragment

(76) Inventors: Gary Allen Wittert, St. Georges (ZA); Christopher Jan Belyea, Elsternwick (AU)

Correspondence Address: KLARQUIST SPARKMAN, LLP, 121 SW SALMON STREET, SUITE 1600, PORTLAND, OR, 97204, US

(21) Appl. No.: 10/512,959

(22) PCT Filed: May 2, 2003
(86) PCT No.: PCT/AU03/00521

§ 371 Date: Oct. 27, 2004

(30) Foreign Application Priority Data
May 3, 2002 (AU) PS 2101
Feb. 27, 2003 (AU) 2003900899

(51) Int. Cl. A61K 38/27

(52) U.S. Cl. 514/12

(57) ABSTRACT

This invention relates to the prevention and treatment of depression and similar mood disorders in mammals, especially humans. In particular, the invention relates to methods for elevating mood in a mammal, comprising administering to the mammal a therapeutically effective amount of a C terminal growth hormone fragment.

This invention relates to the prevention and treatment of depression and similar mood disorders in humans. In particular, the invention relates to a method for alleviating depression or dysphoria.

### BACKGROUND OF THE INVENTION

All references, including any patents or patent applications, cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents forms part of the common general knowledge in the art, in Australia or in any other country.

In Australian patent No. 693478 by Monash University, we described the use of a peptide derived from the carboxyl-terminal sequence of human growth hormone, or corresponding regions from growth hormone of other mammalian species, for the control of obesity. This region of growth hormone has the ability to modulate lipid metabolism. In particular, a synthetic peptide corresponding to amino acid residues 177-191 of the human growth hormone sequence (hereinafter referred to as hGH 177-191) was found to reduce body weight gain and adipose tissue mass in a model system for obesity, the C57Bl/6J (Ob/Ob) mouse. A subsequent application, PCT/AU98/00724 by Metabolic Pharmaceuticals Ltd, discloses analogues of the hGH177-191 peptide which share this activity. The entire disclosures of AU693478 and PCT/AU98/00724 are incorporated herein by this reference.

In our application No. PCT/AU00/01362 (WO01/33977) we also disclose the surprising oral activity of such peptides.

Intact growth hormone has been shown in several studies to have positive effects on mood in patients suffering from growth hormone deficiency. It has become increasingly apparent that the growth-hormone deficient state is accompanied by lower than normal perceived quality of life and tendency to dysphoria or depression, in addition to abnormal fat metabolism (references 1-24).

The first human clinical trials of AOD9604 (Tyr-hgH 177-191) have now been performed, and it has been surprisingly found that after a single dose, AOD9604 in several patients causes mild to moderate euphoria, which is characteristic of the mood-improving properties of the intact growth hormone. It has also been found that after single or multiple oral doses, AOD9604 improves perceived quality of life, as measured by standard questionnaires. The inventors therefore believe that as well as retaining the fat metabolic properties of the intact hormone, C-terminal growth hormone fragments including AOD9604 also retain the mood-improving properties of the intact hormone.

### SUMMARY OF THE INVENTION

In accordance with a broad aspect of the invention there is provided a method of elevating mood in a mammal, comprising administering to the mammal a therapeutic amount of a C terminal growth hormone fragment.

For the purposes of this specification, the term “C-terminal growth hormone fragment” is to be understood to mean a polypeptide fragment from the carboxy-terminal region of the amino acid sequence of a mammalian growth hormone, which has one or more of the following biological activities:
(a) ability to reduce body weight gain and adipose tissue mass in a homologous mammal,
(b) ability to reduce lipogenic activity, and
(c) ability to stimulate lipolysis.

Preferably the growth hormone fragment comprises at least the disulphide-bonded loop of a mammalian growth hormone.

The term “growth hormone fragment” also encompasses peptides which are analogues of the native carboxy-terminal sequences of mammalian growth hormones, provided that the analogue retains one or more of the biological activities referred to above. Such analogues may be derived from natural sources, produced by recombinant DNA technology, or synthesised using conventional peptide synthetic methods. Such peptides synthetic methods are to be understood to include combinatorial methods. Preferably such analogues include a disulphide bond which confers a cyclic configuration on the peptide. In particular, all of the active peptides disclosed in AU 693478 and PCT/AU98/00724 are to be understood to be within the scope of this invention.

Preferably the C-terminal growth hormone fragment comprises amino acids 182-189 (hGH 182-189), more preferably amino acid 177-191 of human growth hormone (hGH 177-191). Even more preferably the C-terminal growth hormone fragment is the analogue AOD9604, Tyr-hGH 177-191. However, it will be clearly understood that the invention is also applicable to growth hormone fragments derived from growth hormones of other mammalian species, including but not limited to those of domestic mammals such as cattle, sheep, pigs and horses, companion animals such as cats and dogs, and zoo animals including felids, canids, and non-human primates. There is strong conservation of the sequence of this region of growth hormone across species, as set out in PCT/AU98/00724 and references cited therein.

The growth hormone fragment may also be conjugated to a fusion partner to enable easier biosynthesis and/or delivery. It may be incorporated in a conventional pharmaceutical composition, or may be present in a genetically-modified food, such as disclosed in WO 01/33997.

The growth hormone fragment may be administered via any suitable route, including oral, buccal, sublingual, intranasal, inhalation, transdermal or intravenous delivery.

Preferably the growth hormone fragment is administered in a pharmaceutical composition for intravenous, subcutaneous or oral delivery. The dosing interval may be once per week, once per day or continuous time release.

Preferably the mammal is suffering from a mood disorder such as depression, dysphoria, anxiety, or social phobia; this may arise from a variety of causes. More preferably the mammal is also growth hormone-deficient and/or obese.

The mammal may be a human, or may be a domestic or companion animal. While it is particularly contemplated that the compounds of the invention are suitable for use in medical treatment of humans, they are also applicable to veterinary treatment, including treatment of companion animals such as dogs and cats, and domestic animals such as horses, cattle and sheep, or zoo animals such as non-human primates, felids, canids, bovids, and ungulates.

Preferably the mammal is a human. The human may be a child or an adult.

Methods and pharmaceutical carriers for preparation of pharmaceutical compositions are well known in the art, as set out in textbooks such as Remington's Pharmaceutical Sciences, 20th Edition, Williams and Williams, Pennsylvania, USA (2000).

The compounds and compositions of the invention may be administered by any suitable route, and the person skilled in the art will readily be able to determine the most suitable route and dose for the condition to be treated. Dosage will be at the discretion of the attendant physician or veterinarian, and will depend on the nature and state of the condition to be treated, the age and general state of health of the subject to be treated, the route of administration, and any previous treatment which may have been administered.

The carrier or diluent, and other excipients, will depend on the route of administration, and again the person skilled in the art will readily be able to determine the most suitable formulation for each particular case.

For the purposes of this specification it will be clearly understood that the word “comprising” means “including but not limited to”, and that the word “comprises” has a corresponding meaning.

### BRIEF DESCRIPTION OF THE FIGURES

FIG. 1 shows the results of assessment of mood using the Nottingham Health Profile Questionnaire in patients from a Phase 2A single dose oral trial. This questionnaire is geared towards negative mood assessment, meaning that a positive numerical result indicates a ‘less happy’ state, and a negative value indicates a ‘happy’ state. The x axis shows fractional change in Nottingham health profile results and the y axis indicates increasing dose of AOD9604. White blocks represent results over all patients tested and black bocks represent results from all non-zero patients tested.

FIGS. 2 and 3 show the results of assessment of mood using the SF-36 Quality of Life Questionnaire in patients from a Phase 2A multiple oral dose escalation trial. This questionnaire yields only positive numerical results, with lower values indicating unhappier and higher values indicating happier.

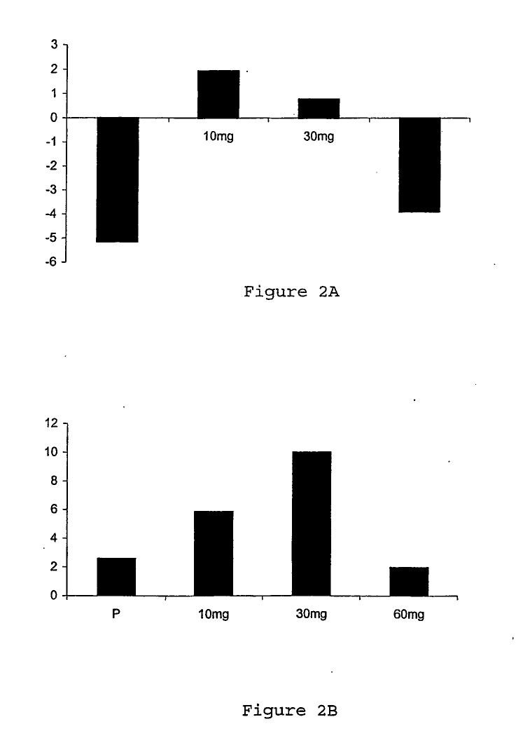

In FIGS. 2A and 2B the x axes represent change in the SF-36 score and the y axes indicate increasing dose of AOD9604. FIG. 2A shows changes in SF-36 questionnaire results at day 8 compared to pre-dose and FIG. 2B shows changes in SF-36 questionnaire results at day 14 compared to pre-dose.

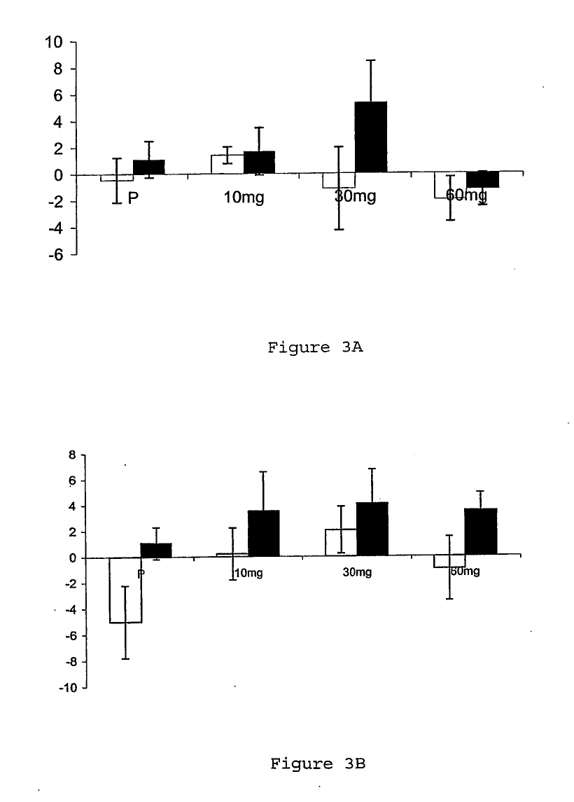

In FIGS. 3A and 3B the x axes represent change in the SF-36 score and the y axes indicate increasing dose of AOD9604. The questionnaire was separated into physical (FIG. 3A) and mental (FIG. 3B) aggregates. White blocks represent results for day 8 compared to day 0 and black blocks represent results for day 14 compared to day 0.

### DETAILED DESCRIPTION OF THE INVENTION

The invention will now be described by way of reference only to the following non-limiting example.

An amino acid sequence variant of the growth hormone fragment defined above is included within the scope of the invention, provided that it is functionally active. As used herein, the terms “functionally active” and “functional activity” in reference to the growth hormone fragment means that the growth hormone fragment is able to alter fat metabolism.

Amino acid sequence variants include deletions, insertions or substitutions of amino acid residues within the growth hormone fragment amino acid sequence set out above. Any combination of deletion, insertion, and substitution may be made to arrive at an amino acid sequence variant of the growth hormone fragment, provided that the variant possesses the desired functional characteristics described herein.

If such substitutions do not result in a change in functional activity, then more substantial changes, denoted exemplary substitutions in Table 1, or as further described below in reference to amino acid classes, may be introduced, and the resulting variant growth hormone fragment analyzed for functional activity.

TABLE 1

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro | pro |
| His (H) | asn; gln; lys; arg | arg |
| Ile (I) | leu; val; met; ala; phe; | leu |
| | norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala | leu |
| Pro (P) | gly | gly |
| Ser (S) | thr | thr |
| Thr (T) | ser | ser |
| Trp (W) | tyr | tyr |
| Tyr (Y) | trp; phe; thr; ser | phe |
| Val (V) | ile; leu; met; phe; | leu |
| | ala; norleucine | |

As used herein, the terms “therapeutically effective amount” and “therapeutic amount” are synonymous, and mean an amount of a growth hormone fragment of the present invention effective to yield a desired therapeutic response.

The specific therapeutically effective amount will obviously vary with such factors as the particular condition being treated, the type of mammal being treated, the physical condition and clinical history of the mammal, the duration of the treatment, the nature of concurrent therapy (if any), and the specific formulations employed and the structure of the growth hormone fragment.

As used herein, a “pharmaceutical carrier” is a pharmaceutically acceptable solvent, suspending agent, excipient or vehicle for delivering the growth hormone fragment and/or pharmaceutically-active agent to the subject. The carrier may be liquid or solid, and is selected with the planned manner of administration in mind.

The growth hormone fragment may be administered orally, sublingually, buccally, transdermally, topically, or parenterally in dosage unit formulations containing conventional non-toxic pharmaceutically acceptable carriers, adjuvants, and vehicles. The term parenteral as used herein includes subcutaneous, intravenous, intramuscular, intrathecal, intracranial, injection or infusion techniques.

The invention also provides suitable topical, oral, aerosol, and parenteral pharmaceutical formulations for use in the novel methods of treatment according to the present invention. Oral dosage forms may be suitable for sublingual or buccal administration The growth hormone fragment of the invention may be administered orally as tablets, aqueous or oily suspensions, lozenges, troches, powders, granules, emulsions, capsules, syrups or elixirs. The composition for oral use may contain one or more agents selected from the group of sweetening agents, flavouring agents, colouring agents and preserving agents in order to produce pharmaceutically elegant and palatable preparations. The tablets contain the active ingredient in admixture with non-toxic pharmaceutically acceptable excipients which are suitable for the manufacture of tablets.

These excipients may, for example, be inert diluents, such as calcium carbonate, lactose, calcium phosphate or sodium phosphate; granulating and disintegrating agents, such as corn starch or alginic acid; binding agents, such as starch, gelatin or acacia; or lubricating agents, such as magnesium stearate, stearic acid or talc. The tablets may be uncoated, or may be coated by known techniques to delay disintegration and absorption in the gastrointestinal tract and thereby provide a sustained action over a longer period. For example, a time-delay material such as glyceryl monostearate or glyceryl distearate may be employed. Coating may also be performed using techniques described in the U.S. Pat. Nos. 4,256,108; 4,160,452; and 4,265,874 to form osmotic therapeutic tablets for control release.

For in vivo application, the growth hormone fragment can be administered parenterally by injection or by gradual perfusion over time. Administration may be intravenous, intra-arterial, intraperitoneal, intramuscular, subcutaneous, or transdermal. Preparations for parenteral administration include sterile aqueous or non-aqueous solutions, suspensions, and emulsions. Examples of non-aqueous solvents include propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles include fluid and nutrient replenishers, electrolyte replenishers such as those based on Ringer's dextrose, and the like. Preservatives and other additives may also be present, such as anti-microbials, anti-oxidants, chelating agents, growth factors and inert gases and the like.

Generally, the terms “treating”, “treatment” and the like are used herein to mean affecting a subject, tissue or cell to obtain a desired pharmacological and/or physiological effect. The effect may be prophylactic in terms of completely or partially preventing a mood disorder involving depression, anxiety or social phobia (collectively referred to herein as “the disease”), or a sign or symptom thereof, and/or may be therapeutic in terms of a partial or complete cure of such a condition. “Treating” as used herein covers any treatment of, or prevention of disease in a mammal, particularly a human, and includes preventing the disease from occurring in a subject who may be predisposed to the disease, but has not yet been diagnosed as having it; inhibiting the disease, ie., arresting its development; or relieving or ameliorating the effects of the disease, ie., cause regression of the effects of the disease.

The invention includes various pharmaceutical compositions useful for ameliorating disease. The pharmaceutical compositions according to one embodiment of the invention are prepared by bringing a growth hormone fragment, analogue, derivatives or salts thereof and one or more pharmaceutically-active agents or combinations of growth hormone fragment and one or more pharmaceutically-active agents into a form suitable for administration to a subject using carriers, excipients and additives or auxiliaries.

Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 20th ed. Williams & Wilkins (2000) and The British National Formulary 43rd ed. (British Medical Association and Royal Pharmaceutical Society of Great Britain, 2002; http://bnf.rhn.net), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed., 1985).

The pharmaceutical compositions are preferably prepared and administered in dosage units. Solid dosage units include tablets, capsules and suppositories. For treatment of a subject, depending on activity of the compound, manner of administration, nature and severity of the disorder, age and body weight of the subject, different daily doses can be used. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The pharmaceutical compositions according to the invention may be administered locally or systemically in a therapeutically effective dose. Amounts effective for this use will, of course, depend on the severity of the disease and the weight and general state of the subject. Typically, dosages used in vitro may provide useful guidance in the amounts useful for in situ administration of the pharmaceutical composition, and animal models may be used to determine effective dosages for treatment of the cytotoxic side effects. Various considerations are described, eg., in Langer, Science, 249: 1527, (1990). Formulations for oral use may be in the form of hard gelatin capsules wherein the active ingredient is mixed with an inert solid diluent, for example, calcium carbonate, calcium phosphate or kaolin. They may also be in the form of soft gelatin capsules wherein the active ingredient is mixed with water or an oil medium, such as peanut oil, liquid paraffin or olive oil.

Aqueous suspensions normally contain the active materials in admixture with excipients suitable for the manufacture of aqueous suspension. Such excipients may be suspending agents such as sodium carboxymethyl cellulose, methyl cellulose, hydroxypropylmethylcellulose, sodium alginate, polyvinylpyrrolidone, gum tragacanth and gum acacia; dispersing or wetting agents, which may be (a) naturally occurring phosphatide such as lecithin; (b) a condensation product of an alkylene oxide with a fatty acid, for example, polyoxyethylene stearate; (c) a condensation product of ethylene oxide with a long chain aliphatic alcohol, for example, heptadecaethylenoxycetanol; (d) a condensation product of ethylene oxide with a partial ester derived from a fatty acid and hexitol such as polyoxyethylene sorbitol monooleate, or (e) a condensation product of ethylene oxide with a partial ester derived from fatty acids and hexitol anhydrides, for example polyoxyethylene sorbitan monooleate.

The pharmaceutical compositions may be in the form of a sterile injectable aqueous or oleaginous suspension. This suspension may be formulated according to known methods using suitable dispersing or wetting agents and suspending agents such as those mentioned above. The sterile injectable preparation may also a sterile injectable solution or suspension in a non-toxic parenterally-acceptable diluent or solvent, for example, as a solution in 1,3-butanediol. Among the acceptable vehicles and solvents which may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid find use in the preparation of injectables.

The growth hormone fragment may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Dosage levels of the growth hormone fragment of the present invention will usually be of the order of about 0.5 mg to about 20 mg per kilogram body weight, with a preferred dosage range between about 0.5 mg to about 10 mg per kilogram body weight per day (from about 0.5 g to about 3 g per patient per day). The amount of active ingredient which may be combined with the carrier materials to produce a single dosage will vary, depending upon the host to be treated and the particular mode of administration. For example, a formulation intended for oral administration to humans may contain about 5 mg to 1 g of an active compound with an appropriate and convenient amount of carrier material, which may vary from about 5 to 95 percent of the total composition. Dosage unit forms will generally contain between from about 5 mg to 500 mg of active ingredient.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

The growth hormone fragment may additionally be combined with other compounds to provide an operative combination. Any chemically compatible combination of pharmaceutically-active agents is within the scope of the invention, provided that the combination does not eliminate the activity of the growth hormone fragment of this invention.

An effective amount of the growth hormone fragment to be employed therapeutically will depend, for example, upon the therapeutic objectives, the route of administration, and the condition of the subject. Accordingly, it will be necessary for the therapist to titrate the dosage and modify the route of administration as required to obtain the optimal therapeutic effect. A typical daily dosage might range from about 1 mcg/kg to up to 1 mg/kg or more, depending on the mode of delivery.

### EXAMPLE 1

### Elevation of Mood in Humans after Administration of AOD9604

The objective of this study was to characterise the safety, tolerability and pharmacodynamic effects of AOD9604 following single intravenous administration to obese adult male subjects. The study was performed by CMAX Pty Ltd under contract to Metabolic Pharmaceuticals Ltd.

The study was designed as a double-blind, placebo-controlled, 4×4 Williams Latin Square design, intravenous dose study included twenty four (obese Body mass index (BMI) of ≧35 kg/m² and a waist circumference of ≧110 cm) adult subjects with each subject receiving active study drug on each of three occasions and placebo on one occasion. Treatment allocation was randomised according to a Master Randomisation Schedule.

The study was conducted over 5 weeks with treatment commencing for Period 1 on 8 Nov. 2001. In order to facilitate subject recruitment, subjects were divided into two groups, Group A (Subjects 01-12) and Group B (Subjects 13-24). Group B subjects, with the exception of Subject 23, commenced treatment two days later than Group A subjects. One subject, Subject 23, commenced treatment a further one week later than Group B subjects. Exit Evaluations were conducted on 6 and 10 December 2001 for all subjects. In each of the four study periods there was a full day in-clinic stay and two overnight stays. There were 7 days between study drug administration in each period.

All subjects enrolled in the study attended the clinical facility within 14 days of initial dose administration to undergo a screening examination to determine their eligibility to participate in the study. The following assessments were made during the Screening Visit: demographics (including measurement of height and weight for the determination of BMI and waist circumference), medical history, drug history, prior and concomitant medications, collection of blood sample (to be tested for routine clinical chemistry, haematology, insulin, cholesterol, Hepatitis B surface antigen, Hepatitis C and HIV antibodies), collection of a urine sample (for detection of drug and alcohol abuse and for urinalysis and microscopy), physical examination including ECG and vital sign measurements. Subjects were also required to complete a Food and Activity Diary, to provide information regarding their eating habits and calorific intake.

On the day prior to dosing for the first period, subjects were admitted to the clinical facility at approximately 1800 hours. The inclusion and exclusion criteria were reviewed by the Principal Investigator, or Co-investigator and written informed consent was obtained. A brief physical examination was conducted and subjects were required to provide a urine specimen for detection of drug and alcohol abuse. An overnight fast commenced following a snack at approximately 2100 hours.

Subjects who provided written informed consent and who met all inclusion/exclusion criteria were randomised to receive each of the four treatments according to the study Master Randomisation Schedule. The subjects were randomised to receive a single dose of 25, 50 and 100 μg/kg AOD9604 and a single dose of placebo (5% mannitol solution) over the four periods of the study. Dose administration was by intravenous infusion, and was conducted over a period of 10 minutes.

During each in-clinic stay, blood samples were collected at specified times both prior to and following dose administration for the determination of pharmacodynamic parameters of non-esterified fatty acids (NEFA), glycerol, glucose and IGF-1. For the purposes of safety monitoring, the physical condition (physical examination, blood pressure, pulse, temperature and general well being) of the subjects was monitored both prior to and at specified intervals during each study period. ECG monitoring was performed prior to the infusion, at the end of the infusion and at specified times following the end of the infusion. Blood samples were also collected for assessment of haematological and biochemical parameters. Subjects were released from the clinic following the 24 hour post-dose blood sample and physical examination. Subjects returned to the clinic for the following period after a 7-day washout period. Within 14 days of the final period (Period 4), an Exit Evaluation was performed, involving the following assessments: physical examination (including ECG monitoring), body height and weight determination, vital sign measurements, questions regarding adverse events, collection of blood samples for the assessment of haematological and biochemical parameters, and collection of a urine sample for urinalysis and microscopy. A further 5 mL blood sample was collected and the serum stored for possible antibody analysis to be done at a later date.

The temperature of the rooms in the clinical facility in which the study activities including dosing, ECGs and blood sampling were performed was monitored using TiniTalk II temperature data loggers.

The overview of the schedule of time and events is presented in Table 2.

TABLE 2
Study Schedule of Events

| | | | | | Exit |
|---|---|---|---|---|---|
| | Screening | | | | Evaluation |
| | Period | Periods 1-4 | | | (Day ≦14 |
| Measurements/ | (Days −14 | Check-in | On-study | On-study | following |
| Treatments | to 1) | (Day −1) | (Day 1) | (Day 2) | Period 4) |
| Inclusion/Exclusion | X | | | | |
| Informed Consent | | X | | | |
| | | (Period 1) | | | |
| Complete Medical History | X | | | | |
| Physical Examination, | X | X | | X^{†} | X^{†} |
| (including height and | | | | | |
| weight) | | | | | |
| Vital Signs | X | X | X | X^{†} | X^{†} |
| Concomitant Medication | X | X | | X^{†} | |
| Urine Drug and Alcohol | X | X | | | |
| Screen | | | | | |
| HIV Test | X | | | | |
| Hepatitis B Antigen Test | X | | | | |
| Electrocardiogram | X | | X | X^{†} | X^{†} |
| Haematology | X | | X | X^{†} | X^{†} |
| Biochemistry | X | | X | X^{†} | X^{†} |
| Study Drug Administration | | | X | | |
| Blood Sample Collection | | | X | X | |
| for PD Analyses* | | | | | |
| AE Monitoring | | X | X | X | X |

^{†}Taken prior to discharge from the clinical facility.
*PD analyses included glycerol, NEFA, blood glucose and IGF-1.

The study was designed as a double-blind, placebo-controlled, 4×4 Williams Latin Square design, intravenous dose study planned to include twenty four (24) obese (BMI of ≧35 kg/m2 and a waist circumference of ≧110 cm) adult subjects, with each subject receiving active study drug on each of three occasions and placebo on one occasion. The study design appropriately met the study objective, which was to characterise the safety, tolerability and pharmacodynamic effects of AOD9604 in this group of subjects.

Subjects were eligible to participate in the study only if all the following entry criteria were met:
were males aged 18-50 years inclusive, at the time of enrolment;
had a Body Mass Index (BMI) ≧35 kg/m2 with a waist circumference ≧110 cm determined by measuring the narrowest point between the bottom of the ribs and the top of the iliac crest in the mid axillary line;
were healthy (determined by a medical history with particular attention to (i) a drug history identifying any known drug allergies and the presence of drug abuse; (ii) any chronic use of medication; and (iii) a thorough review of body systems. This was also determined by having no clinically significant findings on the physical examination, which includes an electrocardiogram (ECG));
had adequate venous access in their left or right arm to allow collection of a number of blood samples via a venous cannula;
were fluent in the English language;
had voluntarily given written informed consent to participate in the study.

Subjects were not eligible for inclusion in this study if any of the following criteria were met:
had a history of severe or multiple allergies, severe adverse drug reaction or leucopaenia or a known hypersensitivity to lignocaine/lidocaine or all surgical dressings which may have been used in the study procedures;
had any evidence of organ dysfunction, or laboratory values considered to be clinically significant by the Medical Officer;
had a history of clinically significant gastrointestinal, hepatic, renal, cardiovascular, dermatological, immunological, respiratory, endocrine, oncologic, neurological, metabolic, psychiatric disease or haematological disorders; or a history of tuberculosis, epilepsy, diabetes or glaucoma;
had a history of intracranial hypertension, thyroid disease, or a strong family history of diabetes;
had a history of asthma during the past 10 years;
had a fasting blood glucose level of ≧6.0 mmol/L;
had hypertension defined as a systolic blood pressure of >140 mmHg and/or a diastolic blood pressure of >90 mmHg;
had a creatinine clearance of less than 75 mL/min;
had abnormal liver function tests defined as higher than twice the upper limit of normal;
had a history of acute or chronic metabolic acidosis, including diabetic ketoacidosis;
had a history of abnormal bleeding tendencies or thrombophlebitis unrelated to venepuncture or intravenous cannulation;
had a history of Hepatitis B, a positive test for Hepatitis B surface antigen, a history of Hepatitis C, a positive test for Hepatitis C antibody, a history of HIV infection or demonstration of HIV antibodies;
were smokers who smoked more than 5 cigarettes, or equivalent per day;
were regular drinkers of more than four (4) units of alcohol daily (1 unit=300 mL beer, 1 glass wine, 1 measure spirit) and who may have had difficulty abstaining from alcohol during the 48 hours prior to dose administration and until completion of blood sampling in each study period;
had a history of or current evidence of, abuse of alcohol or any drug substance, licit or illicit; or positive urine drug and alcohol screen for drugs of abuse and alcohol;
had difficulty in abstaining from prescription medication or over-the-counter (OTC) medication (except occasional paracetamol) within 14 days, or vitamin supplements within 4 days prior to the initial dose administration and for the duration of the study;
would consume more than 5 cups or equivalent of caffeine per day;
had a history of any psychiatric illness which may have impaired the ability to provide written informed consent;
were poor compliers or unlikely to attend;
had received any drug as part of a research study within 30 days of initial dose administration in this study;
had donated blood or blood products within 12 weeks before the initial study dose; or
were dieting to lose weight or participating in a weight reduction program.

The AOD9604 preparation for this study (Lot number 200-01-001) was manufactured by Formatech Incorporated, Andover, Mass., USA in accordance with the principles of Good Manufacturing Practice. The drug was provided in clear glass vials, each vial containing 5 mg AOD9604 as a lyophilised powder. The drug was stored at 2-8° C. in a secure and temperature controlled refrigerated for 11 days at CMAX before being transferred to the Royal Adelaide Hospital Pharmacy where the drug was prepared for clinical dosing.

There were four study treatments, and each subject received each of the four treatments on four separate occasions, separated by a 7-day washout period.

The four treatments were as follows:

Treatment A: A single dose of 25 μg/kg AOD9604 administered intravenously via an infusion pump over a 10 minute period.

Treatment B: A single dose of 50 μg/kg AOD9604 administered intravenously via an infusion pump over a 10 minute period.

Treatment C: A single dose of 100 μg/kg AOD9604 administered intravenously via an infusion pump over a 10 minute period.

Treatment D: A single dose of placebo (5% mannitol solution) administered intravenously via an infusion pump over a 10 minute period.

The Royal Adelaide Hospital Pharmacist selected the appropriate dose level for each subject for each period of the study by following the Master Randomisation Schedule. The subject's body weight at the time of screening was used to calculate the amount of AOD9604 study drug powder required to achieve the desired concentration of 25, 50 or 100 μg/kg in a final volume of 45 mL. The required amount of AOD9604 was reconstituted with sterile water for intravenous injection, made to a 45 mL volume using a 5% mannitol solution and filtered through a 50 micron (Sartorius) single-use filter. The volume was drawn into a 50 mL syringe (Becton Dickinson) using a sterile needle and 20 mL was infused intravenously into the subject via an infusion pump (Graseby 3200 infusion pump with Tuta minimum volume extension tubing) over a 10 minute period. For subjects randomised to receive placebo, a 45 mL volume of 5% mannitol was prepared and 20 mL administered.

Following dosing, an aliquot of the remaining volume of the drug formulation contained in each syringe was dispensed into pre-weighed vials containing aqueous acetic acid (2% v/v) and refrigerated, as per Amendment 1 of the Huntingdon Life Sciences Protocol for Achieved Concentration Analysis of Formulations. Following each study period, the aliquots for subject Groups A and B were sent to Huntingdon Life Sciences Ltd, Cambridgeshire, England for analysis of AOD9604 concentration.

The study was designed to have 24 subjects complete the study. The Master Randomisation Schedule was generated according to a 4×4 Williams Latin Square design. This design results in four possible sequences of treatment allocation: ABCD, BDAC, CADB and DCBA, and subjects were randomly assigned to one of those sequences.

Subjects who provided written informed consent and who met all inclusion and exclusion criteria were assigned a randomisation number. The randomisation number and subject initials were used as the subject's identification code on all study documents and materials.

The selection of 25, 50 and 100 μg/kg doses of AOD9604 used in this study was based on the results from a previous Phase I study, conducted from February-June 2001, in healthy lean males using a dose range of 25-400 μg/kg AOD9604. A pharmacodynamic response was seen within this dose range, but the response diminished as the dose increased to 200 μg/kg and higher. As there had been no safety problems associated with the drug and a response had been shown at 50 μg/kg, it was decided that the dose range of 25-100 μg/kg be chosen for this study in obese male subjects.

Each subject received one of each of three doses of the active study drug, AOD9604 (25, 50, and 100 μg/kg) on three occasions and a placebo on one occasion. The doses were administered as intravenous infusions of 10 minute duration with a wash-out period of 7 days between each consecutive treatment. Subjects were not permitted to eat or drink anything, except for water, for at least 10 hours prior to and 2 hours following administration of the dose. Meals were timed in relation to the end of dosing, as follows: Snack (2 hours and 5 minutes), Lunch (4 hours and 5 minutes), Dinner (10 hours and 5 minutes) and Snack (13 hours and 5 minutes).

This study was designed to ensure that the subjects, Principal Investigator, Co-investigators, nursing staff, and the monitoring staff were blinded to the study treatments. The original Master Randomisation Schedule, which was generated by the study statistician was delivered to the Royal Adelaide Hospital Pharmacist in a sealed envelope, and a second sealed copy retained at CMAX. A third copy was used for the production of code break envelopes, performed by a nominated CMAX staff member who was not directly involved with any other study-related activities. This nominated person created sealed code break envelopes (one envelope per subject per Period labelled with the subject code and Period number) containing documentation of the assigned treatments for that subject. All envelopes were labelled with the protocol number and a statement to only open in the case of an emergency. The envelopes were retained in the study file at CMAX.

Subjects were instructed to abstain from caffeine or other xanthine-containing products and alcoholic beverages for 48 hours prior to dose administration and until completion of the 24 hour post-dose blood sample in each study period. Subjects were required to maintain their regular diet from one month prior to screening until the Exit Evaluation.

During the conduct of the study, subjects were restricted to their beds for the first 4 hours post-dose, after which time they were allowed to ambulate freely while confined at the clinical facility, although strenuous activity was not permitted. Subjects were instructed not to take or use any recreational drugs (i.e. marijuana) for 30 days prior to initiation and through to the completion of the study. They were also asked to refrain from smoking from midnight prior to check-in admission for each study period.

The menu was identical for each of the 4 study periods, and subjects were instructed to complete all the meals provided.

Subjects were to maintain their regular diet from one month prior to screening until the Exit Evaluation. Prior to study enrolment subjects completed a Food and Activity Diary, provided by the Royal Adelaide Hospital dietician, designed to provide information regarding the regular eating habits of the subjects. Using this information and applying the subjects' age and weight measurement at screening to the Schofield Equation, (Schofield W N, 1985), the dietician was able to plan the menu of meals based on the calorific requirement of the subjects. To ensure that the energy intake was balanced for each subject whilst they were confined in the clinical facility, the subjects were divided into six individual Meal Groups, the calorific intake of which ranged from 2664-5010 kcal, divided into 400 kcal increments. Subjects were not permitted to eat or drink anything except for water for at least 10 hours prior to and 2 hours and 5 minutes following administration of the dose. Further meals were provided at 4 hours and 5 minutes, 10 hours and 5 minutes and 13 hours and 5 minutes post-dose.

Results

Pharmacodynamic parameters of serum non-esterified fatty acids (NEFA) and glycerol were used to monitor efficacy of AOD9604 on fat metabolism in this study. Mean NEFA rose more 2 hours post dose for all AOD9604 doses compared to placebo. The effect was highly significant in the older patients, aged 35+. This was the time point and the measure for which a rise was expected, both from the previous study conducted in lean males, and from previous published work on acute effects of growth hormone injections in humans. Body weight change as measured 1 week after each dose was lower in the treated groups than in those receiving placebo. The older patients, aged 35+(N=13) showed highly significant weight loss compared to placebo at the highest dose of 100 μg/kg compared to placebo. These measurements confirmed the activity of AOD9604 on fat metabolism and its potential as an obesity drug.

A surprising finding was that euphoria was experienced by 5/23 (21.7%) subjects, only in the treatment periods when study drug was administered. Five of 23 subjects (21.7%) reported a feeling of mild or moderate euphoria following administration of active drug. The onset of the event ranged from 21 hours to 4 days post drug infusion, and lasted from 1.5 hours up to 7 days. One subject experienced 2 episodes, and another subject experienced 3 episodes of euphoria, ie. in the latter case, euphoria was reported following every active drug infusion. Euphoria was not reported in any subject who received placebo.

The inventors believe that this result suggests that AOD9604 retains the mood-improving properties of the intact hGH.

### EXAMPLE 2

### Double-Blind Single Dose Oral Administration Study

The objective of this study was to characterise the safety, tolerability, pharmacodynamic and pharmacokinetic effects of AOD9604 following single oral administration to obese adult male subjects.

The study was a double-blind, placebo controlled, 4×4 Williams Latin Square design, single oral dose study, planned to include 16 subjects. Each subject received active study drug on each of three occasions and placebo on one occasion. There was a washout period of 14 days between the doses administered in each study period.

Eligibility criteria were the same as for Example 1, except that the age range of the subjects was 35 to 60 years of age inclusive.

There were four study treatments, and each subject received each of the four treatments on four separate occasions, separated by a fourteen day wash out period. The dose strengths were as follows:
10 mg (76.9 μg/kg for 130 kg subject);
30 mg (230.8 μg/kg for 130 kg subject);
60 mg (461.5 μg/kg for 130 kg subject); and
placebo.

The active pharmaceutical formulation was Size-0 capsules (Shionogi Qualicaps Co. Ltd.) of 10 mg AOD9604 containing 3.9% AOD9604, 84.1% Mannitol (USP) and 12% PEG3350, USP. Placebo capsules contained excipients only.

Both the active and placebo formulations were manufactured under Good Manufacturing Practice conditions by PolyPeptide Laboratories, Torrance, Calif., USA.

The batch number and results (certificates of analysis) of in vitro tests to validate potency and dissolution characteristics of AOD9604 were made available to the Principal Investigator and the Investigational Drugs Subcommittee prior to initiation of the study.

Each of the following treatments was administered according to a randomised sequence:

Treatment A: 10 mg AOD9604 (one capsule containing active drug+five placebo capsules).

Treatment B: 30 mg AOD9604 (three capsules containing active drug+three placebo capsules).

Treatment C: 60 mg AOD9604 (six capsules containing active drug only).

Treatment D: placebo (0 mg AOD9604) (six capsules containing placebo only).

For each treatment, capsules were administered orally with 240 mL of room temperature water following at least a 12 hour overnight fast. Study personnel inspected the oral cavity to confirm that the subject has ingested the study treatment following each dose.

The subject's treatment for each study period was prepared according to the study Randomisation Schedule. The master randomisation code and individual sealed envelopes was held at CMAX. In situations where the Principal Investigator deemed it necessary for the treatment code for a specific subject to be broken prior to study completion (e.g. due to a serious adverse event), the date, reason for and name of the individual breaking the code was documented.

Otherwise the study was generally performed as described in Example 1.

In the Phase 2A single dose intravenous trial, reported in Example 1, mild euphoria was reported by several patients who received drug and none who received placebo. The patients taking part in this single dose oral treatment were therefore asked to complete the Nottingham Health Profile questionnaire (see Wiklund I. (1990) The Nottingham Health Profile—a measure of health-related quality of life. Scand. J. Prim. Health Care Suppl. 1:15-8) before dose and 7 days after dose.

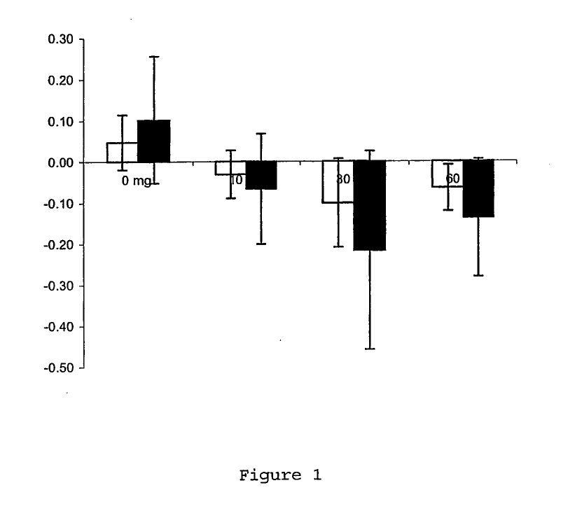

The results showed a worsening of mood with placebo, and improvement of mood at all doses of AOD9604 tested, with the 30 mg dose showing the greatest response (lower is happier). This is illustrated in FIG. 1.

AOD9604 was well tolerated over the oral dose range. One subject was withdrawn from the study following Period 2 due to an adverse event, haematuria, which was deemed unrelated to study drug administration. Another subject was withdrawn following completion of Period 3, due to a Serious Adverse Event, bronchial pneumonia requiring treatment; this was also deemed not to be related to drug administration. There were no observable trends in the incidence of adverse events between the active and placebo treatment groups. There were no clinically significant changes observed in vital sign measurements, electrocardiogram (ECG) measurements, physical examination and clinical laboratory assessments throughout the study.

Non-esterified fatty acid (NEFA) levels were statistically significantly increased compared to placebo at 1, 2 and 4 hours after administration of AOD9604 27.6 mg (p<0.05). A lower response was observed following AOD9604 55.2 mg. A bell-shaped dose response is consistent with observations at intravenous doses up to 300 μg/kg in the Phase 1 trial (conducted in lean patients), and also with in vitro tests on human and animal fat tissue.

### EXAMPLE 3

### Double-Blind Multiple Oral Dose Escalation Study

The objective of this study was to characterise the safety and tolerability and the pharmacodynamic profile of AOD9604 following multiple oral administration to obese adult male subjects.

This was a double-blind, placebo-controlled, 3 period, oral dose, dose escalation study with randomization directed such that each subject received 7 doses of active study drug or placebo over one 7-day period, i.e. one dose per day for 7 days. There were up to three dose levels of AOD9604 and placebo studied in 3 cohorts of 12 subjects each. In each study period, 12 subjects were randomised so that 9 received active study drug and 3 received placebo. There was a period of 7 days between study periods to allow for appropriate safety review and permission to progress to the next dose level.

The eligibility criteria of subjects were as for Example 2, except that the age range was 18 to 60 years of age, and the BMI threshold was ≧30 kg/m², since the primary aim of the study was safety assessment. The restrictions on the subjects and the conduct of the study again were generally as in Example 2.

| | Study Period | | |
|---|---|---|---|
| | 1 | 2 | 3 |
| Proposed Dose | 10 | 30 | 60 |
| (mg/day) | | | |
| Cohort A | Active (n = 9) | | |
| (n = 12 | Placebo (n = 3) | | |
| subjects) | | | |
| Cohort B | | Active (n = 9) | |
| (n = 12 | | Placebo (n = 3) | |
| subjects) | | | |
| Cohort C | | | Active (n = 9) |
| (n = 12 | | | Placebo (n = 3) |
| subjects) | | | |

There were three study periods, and the dose strengths used were 10 mg, 30 mg, 60 mg and placebo, with seven doses per study period. The formulation of AOD9604 and placebo were as for Example 2.

The treatments were carried out as follows, using the same method as in Example 2:

During each study period, each subject was randomized to receive Active Treatment or Placebo (Treatment D).

Treatment A: Study Period 1

10 mg AOD9604 (one capsule containing active drug+five placebo capsules).

Treatment B: Study Period 2

30 mg AOD9604 (three capsules containing active drug+three placebo capsules).

Treatment C: Study Period 3

60 mg AOD9604 (six capsules containing active drug only).

Treatment D: Study Periods 1, 2 and 3

Placebo (0 mg AOD9604) (six capsules containing placebo only).

For each treatment, capsules were administered orally every day for 7 days with 240 mL of room temperature water following at least a 12 hour overnight fast.

The patients were asked to complete the SF-36 Quality of Life Questionnaire (Ware J. J. and Sherbourne C. D. (1992) The MOS 36-item short-form health survey (SF-36). I. Conceptual framework and item selection. Medical Care; 30:473-83) at days 0, 8 and 14. The results are illustrated in FIG. 2, A and B and FIG. 3, A and B. The SF-36 questionnaire is different to the questionnaire used in the Phase 2A intravenous single dose trial of Example 2. SF-36 provides only positive numerical values for mood assessment, with higher values indicating greater happiness, and lower values indicating lower happiness. (The graphs in FIGS. 2 and 3 however do have some negative values because they are representing the change in mood from pre-treatment.) FIGS. 2 and 3 show a similar bell-shaped dose response on mood as that observed in Example 1 (only it is a reversed bell-shape in comparison to Example 1). At 8 days there was an improvement in the mental aggregate for all doses relative to placebo. The improvement at the 10 mg dose was statistically significant (p<0.05). There was an improvement in the mental aggregate scores, relative to placebo, at the 10 mg dose on day 8, and a worsening at the 30 mg and 60 mg doses relative to placebo. At 14 days there was a general increase in mood, probably associated with the return home from the hospital stay, but the relative difference in mood change between the doses was maintained with only the physical aggregate at dose 60 recording a worse result that the placebo.

Body weight difference between the morning of the first day of dosing on day 1 and on the morning after the last day of dosing on day 8 showed clear activity also on the anti-obesity effect, with the same bell-shaped dose response profile as observed, with the maximum effect concentrated in the age 35+ and BMI 35+ group, as predicted, and statistically significant in that subgroup (p<0.05).

As with Example 2, there were no observable trends in the incidence of adverse events between the active and placebo treatment groups, except for an increase in mild gastrointestinal effects, particularly in the hour after dose, in the (ineffective) 60 mg dose group. There were no clinically significant changes observed in vital sign measurements, electrocardiogram (ECG) measurements, physical examination and clinical laboratory assessments throughout the study.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

References cited herein listed on the following pages, and are incorporated herein by this reference.

### REFERENCES

1. Hunt S M, McKenna S P, Doward L C. Preliminary report on the development of a disease-specific instrument for assessing quality of life of adults with growth hormone deficiency. Acta Endocrinologica 1993; 128 (Suppl. 2): 37-40.
2. Hunt S M. Developing a measure of quality of life for adults with growth hormone deficiency. Drug Information Journal. 1994; 28: 3-11.
3. McKenna S P, Doward L C. Quality of Life Assessment of Adults with Growth Hormone Deficiency: Implications for Drug Therapy. PharmacoEconomics 1994; 6 (5): 434-41.
4. Holmes S J, McKenna S P, Doward L C, Shalet S M. Development of a questionnaire to assess the quality of life of adults with growth hormone deficiency. Endocrinology and Metabolism 1995; 2: 63-9.
5. Doward L C, McKenna S P. The development of the AGHDA: A measure to assess quality of life of adults with growth hormone deficiency. Quality of Life Research 1995; 4: 420.
6. McKenna S, Doward L. Measuring quality of life in adults with growth hormone deficiency. Clin Endocrinol (Oxf) 1996 October; 45(4):507-8
7. McKenna S P. Measuring quality of life in growth hormone deficient adults. Proceedings of the International Symposium on GH Deficiency in Adults: Present Status and Future Perspectives (Naples, Dec. 14-16, 1995). Naples: Università “Federico II” Facoltà di Medicina e Chirurgia, 1996.
8. McKenna S P, Doward L C. A specific questionnaire to assess quality of life in GH-deficient adults. Proceedings of the 3^{rd} International hGH Symposium on GHD in Adults. 6 Sep. 1996, Vienna, Austria. Zeist: Medical Forum International, 1997.9-13.
9. McKenna S P, Koppeschaar H P F, Zelissen P M J, Snel Y E M, Whalley D, Doward L C. The impact of replacement GH on the quality of life of patients: Results of a clinical trial in the Netherlands. Endocrinology and Metabolism 1997, 4 (Suppl B): 167.
10. Wirén L, Willhelmsen L, McKenna S P, Hernberg-Ståhl E. The quality of life of adult growth hormone deficient patients in Sweden compared to a random population sample assessed by QOL-AGHDA. Endocrinology and Metabolism 1997, 4 (Suppl B): 167.
11. McKenna S P, Vazquez J A, Pico A, Escobar F, Torres E, Ulied A, Whalley D, Doward L C. The quality of life of adult GHD patients in Spain and the effects of replacement therapy with Genotropin. Endocrinology and Metabolism 1997, 4 (Suppl B): 168.
12. Whalley D, McKenna S P, Wirén L. A disease-specific quality of life measure for use in growth hormone deficient adults (QoL-AGHDA): Assessment by clinical application and Rasch analysis. Quality of Life Research 1998; 7: 672-3.
13. McKenna S P, Doward L C. What is the impact of GH deficiency and GH replacement on quality of life in childhood-onset and adult-onset GH deficiency?. In Monson J P (Ed). Challenges in Growth Hormone Therapy. Oxford: Blackwell Science, 1999: 160-176.
14. McKenna S P, Doward L C, Alonso J, Kohlmann T, Niero M, Prieto L, Wíren L. The QOL-AGHDA: An instrument for the assessment of quality of life in adults with growth hormone deficiency. Quality of Life Research 1999; 8: 373-383.
15. McKenna S P, Whalley D. Assessment of biotechnology drugs: what are the issues? (Letter) Health Policy 1999; 49: 195-7.
16. Wirén L, Whalley D, McKenna S P, Wilhelmsen L. Application of a disease-specific, quality-of-life measure (QoL-AGHDA) in growth hormone-deficient adults and a random population sample in Sweden: validation of the measure by Rasch analysis. Clinical Endocrinology (Oxf) 2000; 52: 143-52.
17. McKenna S P, Doward L, Whalley D. Measuring quality of life in patients with pituitary disease. In. Bengtsson B-Å, Monson J P (Eds.). GH Replacement in Adults: The First Five Years of KIMS. Oxford. Oxford PharmaGenisis Ltd. 2000: 75-87.
18. Murray R D, Shalet S M. Growth hormone: current and future therapeutic applications. Expert Opin Pharmacother. 2000 July; 1(5):975-90. Review.
19. Carroll P V, Littlewood R, Weissberger A J, Bogalho P, McGauley G, Sonksen P H, Russell-Jones D L. The effects of two doses of replacement growth hormone on the biochemical, body composition and psychological profiles of growth hormone-deficient adults. Eur J Endocrinol. 1997 August; 137(2):146-53.
20. Stabler B. Impact of Growth Hormone (GH) Therapy on Quality of Life along the Lifespan of GH-Treated Patients. Horm Res. 2001;56 Suppl 1:55-8.
21. Giusti M, Meineri I, Malagamba D, Cuttica C M, Fattacciu G, Menichini U, Rasore E, Giordano G. Impact of recombinant human growth hormone treatment on psychological profiles in hypopituitary patients with adult-onset growth hormone deficiency. Eur J Clin Invest. 1998 January; 28(1):13-9.
22. Soares C N, Musolino N R, Cunha Neto M, Caires M A, Rosenthal M C, Camargo C P, Bronstein M D. Impact of recombinant human growth hormone (RH-GH) treatment on psychiatric, neuropsychological and clinical profiles of GH deficient adults. A placebo-controlled trial. Arq Neuropsiquiatr. 1999 June; 57(2A):182-9.
23. Birmaher B, Dahl R E, Williamson D E, Perel J M, Brent D A, Axelson D A, Kaufman J, Dorn L D, Stull S, Rao U, Ryan N D. Growth hormone secretion in children and adolescents at high risk for major depressive disorder. Arch Gen Psychiatry. 2000 September; 57(9):867-72.
24. Wiren L, Johannsson G, Bengtsson B A. A prospective investigation of quality of life and psychological well-being after the discontinuation of GH treatment in adolescent patients who had GH deficiency during childhood. J Clin Endocrinol Metab. 2001 August; 86(8):3494-8.

1. A method of elevating mood in a mammalian subject, comprising the step of administering to the subject in need thereof a therapeutically effective amount of a C terminal growth hormone fragment.

2. A method according to claim 1, wherein the growth hormone fragment comprises at least the C terminal disulphide-bonded loop of a mammalian growth hormone.

3. A method according to claim 1, wherein the growth hormone fragment incorporates amino acids 182-189 from human growth hormone.

4. A method according to claim 3, wherein the growth hormone fragment incorporates amino acids 177-191 of human growth hormone.

5. A method according to claim 1, wherein the growth hormone fragment incorporates the amino acids Tyr-hGH 177-191 (AOD9604).

6. A method according to claim 1, wherein the growth hormone fragment is an analogue of human growth hormone.

7. A method according to claim 1, wherein the mammal is a human.

8. A method according to claim 1, wherein the mammal is a companion animal, a domestic animal, or a zoo animal.

9. A method according to claim 1, wherein the growth hormone fragment is administered by oral, buccal, sublingual, intranasal, inhalation, transdermal, subcutaneous or intravenous delivery.

10. A method according to claim 1, wherein the subject is suffering from a mood disorder including depression, dysphoria, anxiety, or social phobia.

11. A method according to claim 10, wherein the subject is growth hormone-deficient or obese.

12-16. (canceled)
